(19) United States
(12) Patent Application Publication
(10) Pub. No.: US 2023/0190976 A1
(43) Pub. Date: Jun. 22, 2023

(54) STERILIZATION SYSTEM AND CONTROL METHOD THEREOF

(71) Applicants: HYUNDAI MOTOR COMPANY, SEOUL (KR); KIA CORPORATION, SEOUL (KR); YOONJIN ELECTRONICS CO., LTD., Gwangmyeong-si (KR)

(72) Inventors: Wansu Park, Gwacheon-si (KR); Tae Hoon LEE, Suwon-si (KR); Sang Hoon PARK, Incheon (KR); Sung Hwan KIM, Incheon (KR); Sang Ki LEE, Gyeonggi-do (KR); Sung Young KIM, Gwangmyeong-si (KR)

(21) Appl. No.: 17/943,995

(22) Filed: Sep. 13, 2022

(30) Foreign Application Priority Data
Dec. 16, 2021 (KR) 10-2021-0181022

(51) Int. Cl. A61L 2/24; A61L 2/10; B60S 3/00

(52) U.S. Cl. CPC A61L 2/24; A61L 2/10; B60S 3/008; A61L 2202/11; A61L 2202/14; A61L 2202/25

(57) ABSTRACT

A sterilization system that sterilizes an interior of a storage compartment of a vehicle, including: at least one lamp configured to irradiate ultraviolet light to the interior of the storage compartment, a sensor configured to detect an open or closed state of the storage compartment, and a controller configured to based on the storage compartment being switched from an open state to a closed state in a state where the sterilization system is turned on, turn on the at least one lamp, count a duration for which the at least one lamp is in an ON state, based on the counted duration reaching a preset duration, turn off the at least one lamp, and based on the storage compartment being switched to the open state in a state where the at least one lamp is turned on, turn off the at least one lamp.

### CROSS-REFERENCE TO RELATED APPLICATION

This application is based on and claims priority under 35 U.S.C. §119 to Korean Patent Application No. 10-2021-0181022, filed on Dec. 16, 2021 in the Korean Intellectual Property Office, the disclosure of which is incorporated herein by reference.

### TECHNICAL FIELD

The disclosure relates to a sterilization system and a control method thereof, and more particularly, to a sterilization system and a control method thereof that may sterilize an interior of a storage compartment of a vehicle.

### BACKGROUND

Various technologies for sterilizing personal items have been being developed in response to an increased interest in personal hygiene due to the SARS-CoV-2 pandemic.

For example, a portable sterilization device capable of sterilizing personal items by ultraviolet (UV) irradiation has been recently used.

However, because the portable sterilization device is too large to carry on a vehicle, the portable sterilization device is required to be disposed in a particular position for use.

### SUMMARY

An aspect of the disclosure provides a sterilization system and a control method thereof that may sterilize a personal item in a storage compartment of a vehicle.

An aspect of the disclosure provides a sterilization system and a control method thereof that may prevent a risk of vehicle occupant’s exposure to ultraviolet in advance.

An aspect of the disclosure provides a sterilization system and a control method thereof that may sterilize a personal item in a storage compartment of a vehicle with optimum energy efficiency.

An aspect of the disclosure provides a sterilization system and a control method thereof that may prevent a sterilization operation from being stopped due to a movement of a vehicle.

An aspect of the disclosure provides a sterilization system and a control method thereof that may allow a vehicle occupant to easily check a state of the sterilization system.

Additional aspects of the disclosure will be set forth in part in the description which follows and, in part, will be obvious from the description, or may be learned by practice of the disclosure.

According to an embodiment of the disclosure, there is provided a sterilization system that sterilizes an interior of a storage compartment of a vehicle, including at least one lamp configured to irradiate ultraviolet light to the interior of the storage compartment, a sensor configured to detect an open or closed state of the storage compartment; and a controller configured to: based on the storage compartment being switched from an open state to a closed state in a state where the sterilization system is turned on, turn on the at least one lamp, count a duration for which the at least one lamp is in an ON state, based on the counted duration reaching a preset duration, turn off the at least one lamp, and based on the storage compartment being switched to the open state in a state where the at least one lamp is turned on, turn off the at least one lamp.

Also, based on the storage compartment being switched to the closed state within a preset period of time after the at least one lamp is turned off due to an opening of the storage compartment in the state where the at least one lamp is turned on, the controller is configured to turn on the at least one lamp and accumulate the counted duration to count the duration for which the at least one lamp is in the ON state.

Also, the controller is configured to reset the counted duration, based on the preset period of time being elapsed after the at least one lamp is turned off due to the opening of the storage compartment in the state where the at least one lamp is turned on.

Also, the controller is configured to when turning on the at least one lamp in a state where the counted duration is not reset, turn on the at least one lamp at a point in time that the storage compartment is switched to the closed state, and when turning on the at least one lamp in a state where the counted duration is reset, turn on the at least one lamp after a delay time from the point in time that the storage compartment is switched to the closed state.

Also, the sterilization system further includes: a power supply configured to supply power supplied from a battery of the vehicle to the sterilization system, wherein the controller is configured to turn off the at least one lamp based on a voltage level of the power supplied from the power supply being out of a preset range.

Also, based on the voltage level returning to within the preset range within a preset period of time after the at least one lamp is turned off due to a decrease in the voltage level below a preset level in the state where the at least one lamp is turned on, the controller is configured to turn on the at least one lamp and accumulate the counted duration to count the duration for which the at least one lamp is in the ON state.

Also, the controller is configured to reset the counted duration, based on the preset period of time being elapsed after the at least one lamp is turned off due to the decrease in the voltage level below the preset level in the state where the at least one lamp is turned on.

Also, the controller is configured to when the at least one lamp is turned off due to an increase in the voltage level above a preset level in the state where the at least one lamp is turned on, maintain the counted duration until the voltage level returns to within the preset range, and based on the voltage level returning to within the preset range, turn on the at least one lamp and accumulate the counted duration to count the duration for which the at least one lamp is in the ON state.

Also, the sterilization system further includes: an indicator configured to provide a visual feedback related to a state of the sterilization system, wherein the controller is configured to control the indicator to provide a first visual feedback based on the voltage level being equal to or less than a first preset level, and control the indicator to provide a second visual feedback based on the voltage level being equal to or greater than a second preset level, the first preset level is a lower limit value of the preset range, the second preset level is an upper limit value of the preset range, and the first visual feedback and the second visual feedback are different from each other.

Also, the at least one lamp includes a plurality of lamps, and the controller is configured to detect a failure state of the plurality of lamps based on a current applied to the plurality of lamps.

Also, the controller is configured to switch the sterilization system to an OFF state, based on at least one lamp of the plurality of lamps being determined as a short-circuited state.

Also, when at least one lamp of the plurality of lamps is determined as an open state and other lamps of the plurality of lamps are determined as a normal state, the controller is configured to adjust the preset duration based on a number of lamps determined as the open state.

Also, the sterilization system further includes: an indicator configured to provide a visual feedback related to a state of the sterilization system, wherein the controller is configured to control the indicator to provide a first visual feedback based on at least one lamp of the plurality of lamps being determined as a short-circuited state, and control the indicator to provide a second visual feedback based on at least one lamp of the plurality of lamps being determined as an open state, and the first visual feedback and the second visual feedback are different from each other.

Also, the sterilization system further includes: an indicator configured to provide a visual feedback related to a state of the sterilization system, wherein the controller is configured to turn on the indicator based on the at least one lamp being turned on.

Also, the sterilization system further includes an inputter configured to receive a user input for switching the sterilization system to an ON state or an OFF state, wherein the inputter is provided inside the storage compartment.

According to an embodiment of the disclosure, there is provided a control method of a sterilization system that sterilizes an interior of a storage compartment of a vehicle, the control method including: based on the storage compartment being switched from an open state to a closed state in a state where the sterilization system is turned on, turning on at least one lamp that irradiates UV to the interior of the storage compartment, counting a duration for which the at least one lamp is in an ON state, based on the counted duration reaching a preset duration, turning off the at least one lamp, and based on the storage compartment being switched to the open state in a state where the at least one lamp is turned on, turning off the at least one lamp.

Also, the control method further includes: based on the storage compartment being switched to the closed state within a preset period of time after the at least one lamp is turned off due to an opening of the storage compartment in the state where the at least one lamp is turned on, turning on the at least one lamp and accumulating the counted duration to count the duration for which the at least one lamp is in the ON state.

Also, the control method further includes resetting the counted duration, based on the preset period of time being elapsed after the at least one lamp is turned off due to the opening of the storage compartment in the state where the at least one lamp is turned on.

Also, the control method further includes: when turning on the at least one lamp in a state where the counted duration is not reset, turning on the at least one lamp at a point in time that the storage compartment is switched to the closed state, and when turning on the at least one lamp in a state where the counted duration is reset, turning on the at least one lamp after a delay time from the point in time that the storage compartment is switched to the closed state.

Also, the control method further includes turning off the at least one lamp, based on a voltage level of power supplied from a power supply being out of a preset range, the power supply configured to supply power supplied from a battery of the vehicle to the sterilization system.

Also, the control method further includes: based on the voltage level returning to within the preset range within a preset period of time after the at least one lamp is turned off due to a decrease in the voltage level below a preset level in the state where the at least one lamp is turned on, turning on the at least one lamp and accumulating the counted duration to count the duration for which the at least one lamp is in the ON state.

Also, the control method further includes resetting the counted duration, based on the preset period of time being elapsed after the at least one lamp is turned off due to the decrease in the voltage level below the preset level in the state where the at least one lamp is turned on.

Also, the control method further includes when the at least one lamp is turned off according to an increase in the voltage level above a preset level in the state where the at least one lamp is turned on, maintaining the counted duration until the voltage level returns to within the preset range, and based on the voltage level returning to within the preset range, turning on the at least one lamp and accumulating the counted duration to count the duration for which the at least one lamp is in the ON state.

Also, the control method further includes controlling an indicator to provide a first visual feedback based on the voltage level being equal to or less than a first preset level; and controlling the indicator to provide a second visual feedback based on the voltage level being equal to or greater than a second preset level, wherein the first preset level is a lower limit value of the preset range, the second preset level is an upper limit value of the preset range, and the first visual feedback and the second visual feedback are different from each other.

Also, the at least one lamp includes a plurality of lamps, and the control method of the sterilization system further includes detecting a failure state of the plurality of lamps based on a current applied to the plurality of lamps.

Also, the control method further includes switching the sterilization system to an OFF state, based on at least one lamp of the plurality of lamps being determined as a short-circuited state.

Also, the control method further includes when at least one lamp of the plurality of lamps is determined as an open state and other lamps of the plurality of lamps are determined as a normal state, adjusting the preset duration based on a number of lamps determined as the open state.

Also, the control method further includes controlling an indicator to provide a first visual feedback based on at least one lamp of the plurality of lamps being determined as a short-circuited state; and controlling the indicator to provide a second visual feedback based on at least one lamp of the plurality of lamps being determined as an open state, wherein the first visual feedback and the second visual feedback are different from each other.

Also, the control method further includes turning on an indicator based on the at least one lamp being turned on.

Also, the control method further includes receiving a user input for switching the sterilization system to an ON state through an inputter provided inside the storage compartment.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a block diagram illustrating a configuration of a sterilization system according to an embodiment;

FIG. 2 is a diagram illustrating an example of a vehicle equipped with a sterilization system according to an embodiment;

FIG. 3 is a diagram illustrating an example of a storage compartment equipped with a sterilization system;

FIG. 4 is a flowchart illustrating an example of a control method of a sterilization system according to an embodiment;

FIG. 5 is a diagram illustrating an operation state of each constituent component of a sterilization system when a storage compartment is not open while sterilization is being performed;

FIG. 6 is a diagram illustrating an operation state of each constituent component of a sterilization system when a storage compartment is open for a relatively short time while sterilization is being performed;

FIG. 7 is a diagram illustrating an operation state of each constituent component of a sterilization system when a storage compartment is open for a relatively long time while sterilization is being performed;

FIG. 8 is a flowchart illustrating another example of a control method of a sterilization system according to an embodiment;

FIG. 9 is a diagram illustrating an operation state of each constituent component of a sterilization system when a voltage level supplied to the sterilization system is switched to a low voltage level while sterilization is being performed;

FIG. 10 is a diagram illustrating an operation state of each constituent component of a sterilization system when a voltage level supplied to the sterilization system is switched to a low voltage level for a relatively short time while sterilization is being performed;

FIG. 11 is a diagram illustrating an operation state of each constituent component of a sterilization system when a voltage level supplied to the sterilization system is switched to a low voltage level for a relatively long time while sterilization is being performed;

FIG. 12 is a diagram illustrating an operation state of each constituent component of a sterilization system when a voltage level supplied to the sterilization system is switched to a high voltage level while sterilization is being performed;

FIG. 13 is a flowchart illustrating still another example of a control method of a sterilization system according to an embodiment; and

FIG. 14 illustrates an example where a sterilization operation duration is changed depending on a number of malfunctioning lamps.

### DETAILED DESCRIPTION

Advantages and features of embodiments, and methods of achieving the same will be clearly understood with reference to the accompanying drawings and the following detailed embodiments. However, the present inventive concept is not limited to embodiments described herein, but may be implemented in various different forms. Embodiments are provided in order to explain the present inventive concept for those skilled in the art. The scope of the present inventive concept is defined by the appended claims.

The terms used herein will be briefly described and embodiments will be described in detail.

Although the terms used herein are selected from among general terms that are currently and widely used in consideration of functions in embodiments, these may be changed according to intentions or customs of those skilled in the art or the advent of new technology. In addition, in a specific case, some terms may be arbitrarily selected by applicants. In this case, meanings thereof will be described in a corresponding description of embodiments. Therefore, the meanings of terms used herein should be interpreted based on substantial meanings of the terms and content of this entire specification, rather than simply the terms themselves.

Throughout this specification, when a certain part “includes” a certain component, it means that another component may be further included not excluding another component unless otherwise defined. Moreover, terms described in the specification such as “part,” “module,” and “unit,” refer to a unit of processing at least one function or operation, and may be implemented by software, a hardware component such as a field-programmable gate array (FPGA) or an application-specific integrated circuit (ASIC), or a combination of software and hardware. However, the terms “part,” “module,” “unit,” and the like are not limited to software or hardware. “Part,” “module,” “unit,” and the like may be configured in a recording medium that may be addressed or may be configured to be reproduced on at least one processor. Therefore, examples of the terms “part,” “module,” “unit,” and the like include software components, object-oriented software components, components such as class components and task components, processes, functions, properties, procedures, subroutines, segments in program codes, drivers, firmware, microcode, circuits, data, databases, data structures, tables, arrays, and variables. The components and the modules may be provided into smaller number of components and modules such that the respective component and modules may be merged in respect to the functionality.

Hereinafter, with reference to the accompanying drawings, embodiments of a sterilization system and a control method thereof will be described in detail so that a person skilled in the art can easily implement the disclosure. In addition, parts irrelevant to description are omitted in the drawings in order to clearly explain exemplary embodiments. In the accompanying drawings, parts that are identical or equivalent to each other will be assigned the same reference numerals, and in the following description of the embodiments, details of redundant descriptions thereof will be omitted.

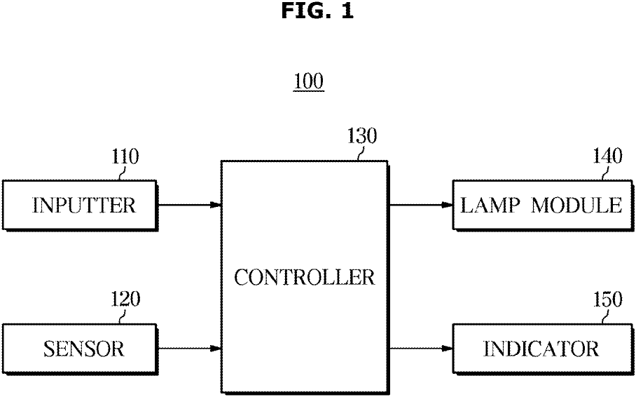

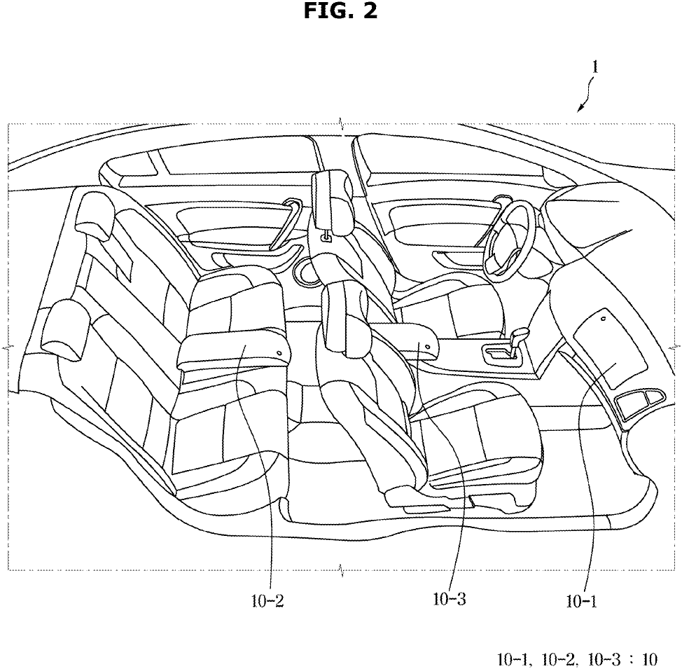

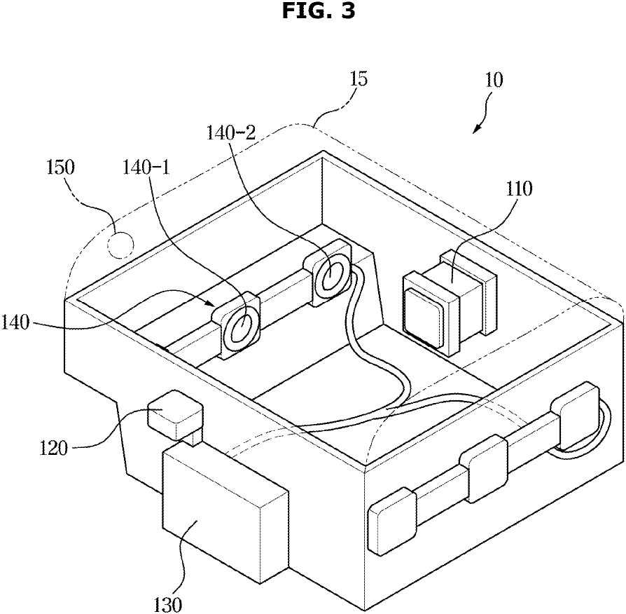

FIG. 1 is a block diagram illustrating a configuration of a sterilization system according to an embodiment. FIG. 2 is a diagram illustrating an example of a vehicle equipped with a sterilization system according to an embodiment. FIG. 3 is a diagram illustrating an example of a storage compartment equipped with a sterilization system.

Referring to FIGS. 1 to 3, a sterilization system 100 according to an embodiment may include an inputter 110, a sensor 120, a controller 130, a lamp module 140, and an indicator 150.

A vehicle 1 may include at least one storage compartment 10 capable of storing a personal item.

An occupant of the vehicle 1 may store the item in the storage compartment 10 by manually opening a cover 15 of the storage compartment 10, placing the item in the storage compartment 10, and then manually closing the cover 15 of the storage compartment 10.

According to various embodiments, the storage compartment 10 may include a first storage compartment 10-1 provided in a dashboard in front of a passenger seat and/or a second storage compartment 10-2 provided between rear seats, and/or a third storage compartment 10-3 provided in a center fascia between the passenger seat and a driver’s seat.

However, positions and the number of storage compartments 10 are not limited thereto, and may vary depending on a type, size, shape, design intention, etc., of the vehicle 1 without limitation.

Hereinafter, for convenience of description, it is assumed that the storage compartment 10 refers to at least one storage compartment 10 provided inside the vehicle 1, for example, the first storage compartment 10-1, the second storage compartment 10-2, and/or the third storage compartment 10-3.

The storage compartment 10 may include a housing for forming an accommodating space therein, and the cover 15 for opening and closing the accommodating space formed by the housing.

The occupant may open and close the storage compartment 10 by opening and closing the cover 15 manually.

Hereinafter, for convenience of description, a state where the accommodating space is covered by the cover 15 is defined as a closed state of the storage compartment 10, and a state where the cover 15 opens the accommodating space is defined as an open state of the storage compartment 10.

The inputter 110 may receive a user input for switching the sterilization system 100 to an ON state or OFF state.

According to an embodiment, the inputter 110 may be implemented as a pushlock switch. The pushlock switch refers to a button-type input device that maintains an ON state or an OFF state according to an occupant’s act of pushing the pushlock switch.

For example, the pushlock switch may form an electrical contact with the sterilization system 100 (e.g., the controller 130) in a pushed state, and form an open circuit in an un-pushed state.

However, an example of the inputter 110 is not limited thereto, and the inputter 110 may include any type of inputter capable of receiving a user input for switching the sterilization system 100 to an ON state or an OFF state.

For example, the inputter 110 may include a touch button, a touch pad, a touch screen, a dial, a stick-type operation device and/or a track ball.

When the inputter 110 is provided outside the storage compartment 10, the occupant is incapable of intuitively grasping what the inputter 110 is for, and also power is wasted by activating the sterilization system 100 through unnecessary manual operations.

According to various embodiments, the inputter 110 may be provided inside the storage compartment 10. Because inputter 110 is provided inside the storage compartment 10, the occupant may operate the inputter 110 only when the occupant desires to sterilize the occupant’s personal item.

For example, the occupant may sterilize the occupant’s item by opening the storage compartment 10, switching the sterilization system 100 to an ON state by operating the inputter 110, and then putting the item in the storage compartment 10 and closing the storage compartment 10.

According to the disclosure, by disposing the inputter 110 for operating an ON/OFF state of the sterilization system 100 inside the storage compartment 10, the occupant may intuitively grasp what the inputter 110 is for and be induced to turn on the sterilization system 100 only when the occupant intends to sterilize the item.

However, a position of the inputter 110 is not limited to the interior of the storage compartment 10, and may be provided outside the storage compartment 10 or be implemented integrally with an electronic component (e.g., an audio, video, navigation (AVN) device) of the vehicle 1.

According to an embodiment, even when the inputter 110 is provided outside the storage compartment 10, the sterilization system 100 may operate the lamp module 140 only when the storage compartment 10 is switched from an open state to a closed state in order to prevent an unnecessary ultraviolet (UV) irradiation.

For example, even when the sterilization system 100 is switched to an ON state through the occupant’s operation of the inputter 110 in a state where the storage compartment 10 is closed, when an opening or closing of the storage compartment 10 after the sterilization system 100 is switched to the ON state is not detected, the lamp module 140 may not be operated.

According to another embodiment, when the inputter 110 is provided outside the storage compartment 10 and the storage compartment 10 is in a closed state at a point in time that the sterilization system 100 is switched to an ON state, the lamp module 140 may be operated.

In order to apply the various examples described above, the occupant of the vehicle 1 may set an operation point in time of the lamp module 140 through a user interface provided in the vehicle 1.

According to various embodiments, when the inputter 110 is implemented as the pushlock switch, a pushed state may be automatically released based on completion of a sterilization operation of the sterilization system 100.

According to the disclosure, by automatically switching the sterilization system 100 to an OFF state after the completion of the sterilization operation, an occupant’s intention may be understood again to proceed with a next sterilization operation. Accordingly, unnecessary UV irradiation may be prevented, energy for irradiating UV may be saved, and a risk of occupant’s exposure to UV may be reduced.

The sensor 120 may include at least one sensor capable of collecting various data required for operating the sterilization system 100.

According to an embodiment, the sensor 120 may include a sensor capable of detecting an open/closed state of the storage compartment 10 (hereinafter, a “first sensor”).

The first sensor may include any type of sensor (e.g., an electronic sensor and/or a mechanical sensor) capable of detecting various types of values that are changed depending on an opening or closing of the cover 15 of the storage compartment 10.

For example, the first sensor may include a hall sensor capable of detecting a magnetic field that varies depending on an opening or closing of the cover 15 of the storage compartment 10.

As another example, the first sensor may include a pressure sensor capable of detecting a pressure generated by closing the cover 15 of the storage compartment 10.

The first sensor may detect whether the storage compartment 10 is open or closed, and transmit information about the open/closed state of the storage compartment 10 to the controller 130.

According to an embodiment, the sensor 120 may include a sensor capable of detecting a failure state of the lamp module 140 (hereinafter, a “second sensor”).

The second sensor may include any type of sensor (e.g., an electronic sensor and/or a mechanical sensor) capable of detecting various types of values that are changed due to a failure of the lamp module 140.

For example, the second sensor may include a current sensor detecting a current flowing through the lamp module 140 and/or a voltage sensor detecting a voltage applied to the lamp module 140.

The second sensor may detect a current value flowing through the lamp module 140 and/or a voltage value applied to the lamp module 140, and transmit the current value flowing through the lamp module 140 and/or the voltage value applied to the lamp module 140 to the controller 130.

The controller 130 may control overall operations of the sterilization system 100. For example, the controller 130 may control the lamp module 140 and/or the indicator 150 based on a state of the sterilization system 100 switched through the inputter 110 and information collected through the sensor 120.

The controller 130 may include at least one memory in which a program for performing the above-described operations and operations to be described below is stored, and at least one processor for executing the stored program.

When the controller 130 includes a plurality of memories and a plurality of processors, the plurality of memories and the plurality of processors may be integrated into one chip or may be physically separated.

According to various embodiments, the controller 130 may be implemented as a micro controller unit (MCU) for controlling the sterilization system 100.

A housing that houses the controller 130 may be provided in one side of the storage compartment 10, but a position of the housing that houses the controller 130 may vary without limitation.

The lamp module 140 may include at least one lamp 140-1 and 140-2 for irradiating UV to the interior of the storage compartment 10. The at least one lamp 140-1 and 140-2 may include a plurality of lamps.

The at least one lamp 140-1 and 140-2 may be provided inside the storage compartment 10 and emit UV to the interior of the storage compartment 10. The at least one lamp 140-1 and 140-2 may be provided on both sides of the inside of the storage compartment 10 to efficiently sterilize items inside the storage compartment 10.

The at least one lamp may include an ultraviolet (UV) light emitting diode (UV LED) for irradiating UV light.

UV light may be divided into UV-A (long wave), UV-B (medium wave) and UV-C (short wave) according to a wavelength, and a wavelength of UV-C for sterilization may be in a range of approximately 100 to 280 nm.

According to an embodiment, the at least one lamp 140-1 and 140-2 may include an UVC LED for irradiating UVC rays.

When UV light directly irradiates a human body, the UV light may harm the human body. Accordingly, the housing and/or cover of the storage compartment 10 may be implemented as a member capable of blocking a leakage of UV.

The lamp module 140 may be turned on or off based on a control signal of the controller 130.

When the lamp module 140 is turned on, all of the at least one lamp is capable of irradiating UV to the interior of the storage compartment 10. When the lamp module 140 is turned off, all of the at least one lamp is capable of stopping the UV irradiation.

The indicator 150 may provide a visual feedback related to a state of the sterilization system 100.

For example, the indicator 150 may include any type of device for providing the visual feedback related to a state of the sterilization system 100 (e.g., a peak indicator such as a tape recorder using an LED plasma display, a tuner signal indicator, and a stereo indicator).

The indicator 150 flashes in a blinking pattern (e.g., ON/OFF cycle and/or a color of blinking light) corresponding to the state of the sterilization system 100 based on the control signal of the controller 130 to visually inform the occupant of the state of the sterilization system 100.

According to an embodiment, the indicator 150 may be provided outside of the storage compartment 10 (e.g., the cover 15 of the storage compartment 10), without being limited thereto. The indicator 150 may be disposed anywhere as long as it enables the occupant to be easily provided with the visual feedback.

The sterilization system 100 may include a power supply (not shown) that supplies power, supplied from a battery of the vehicle 1, to the sterilization system 100.

The power supply may supply power to the controller 130 and/or the sensor 120 and/or the lamp module 140.

According to various embodiments, the controller 130 may transmit and receive various information through a vehicle communication network and a plurality of electronic control units (ECU) provided in the vehicle 1.

For example, the controller 130 may transmit failure information to an electronic component of the vehicle 1 through the vehicle communication network based on detection of a failure of the lamp module 140.

The vehicle communication network may include Ethernet, media oriented systems transport (MOST), FlexRay, controller area network (CAN), local interconnect network (LIN), and the like.

Although the constituent components of the sterilization system 100 have been described, an additional constituent component may be added or the above-described constituent component may be omitted within a general technical scope.

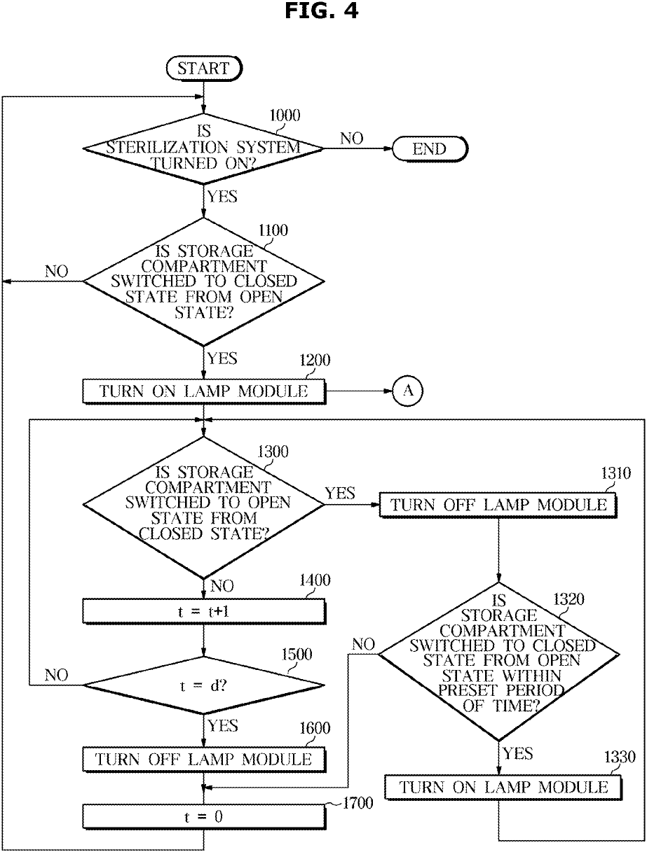

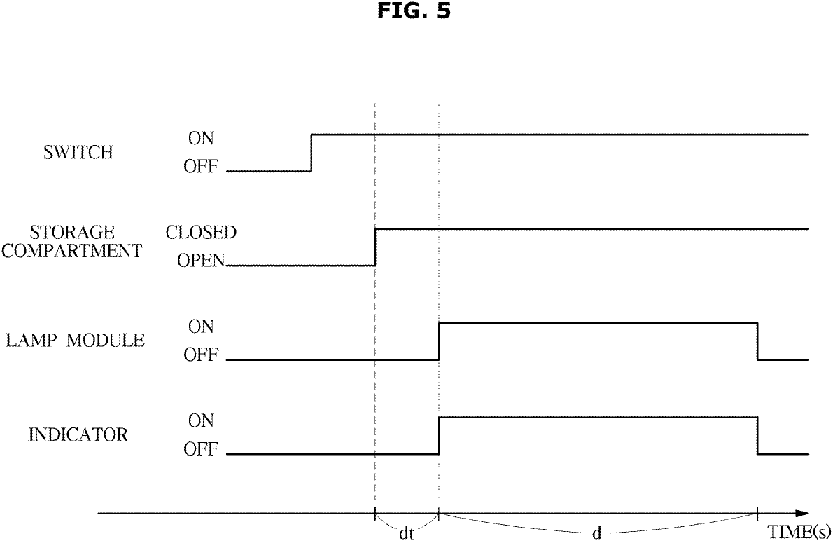

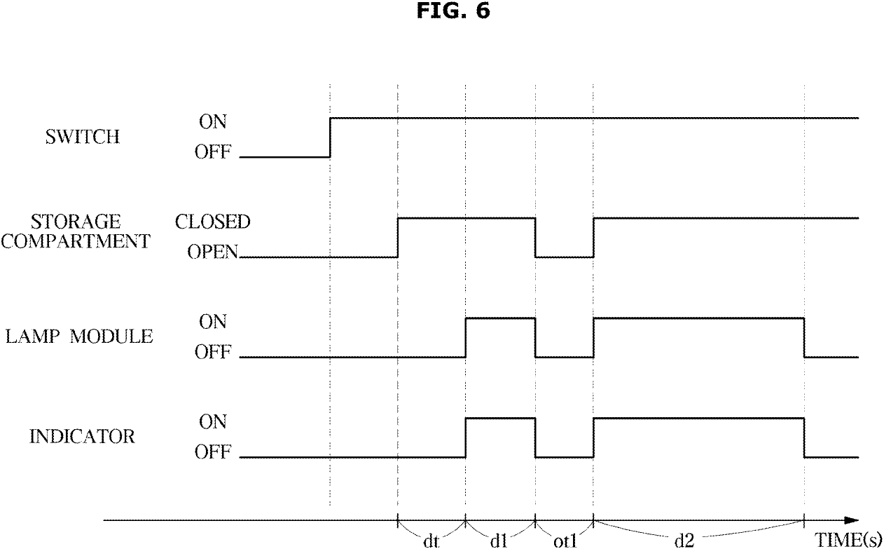

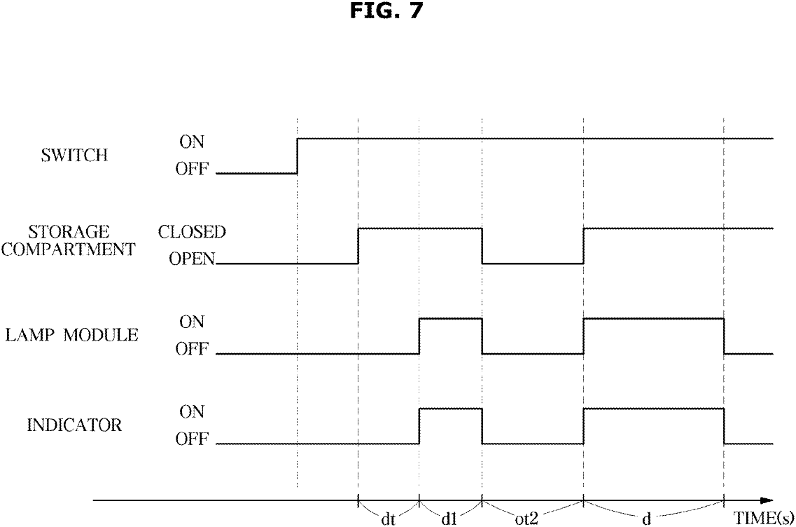

FIG. 4 is a flowchart illustrating an example of a control method of a sterilization system according to an embodiment. FIG. 5 is a diagram illustrating an operation state of each constituent component of a sterilization system when a storage compartment is not open while sterilization is being performed. FIG. 6 is a diagram illustrating an operation state of each constituent component of a sterilization system when a storage compartment is open for a relatively short time while sterilization is being performed. FIG. 7 is a diagram illustrating an operation state of each constituent component of a sterilization system when a storage compartment is open for a relatively long time while sterilization is being performed.

Referring to FIG. 4, the controller 130 may identify whether the sterilization system 100 is turned on (1000).

The controller 130 may determine that the sterilization system 100 is turned on, based on receiving a user input for switching the sterilization system 100 to an ON state through the inputter 110 (Yes in operation 1000).

When the sterilization system 100 is in an OFF state (No in operation 1000), the controller 130 may not perform any operation related to the sterilization system 100. For instance, when the sterilization system 100 is in the OFF state, the controller 130 may not be operated. Also, when at least one sensor included in the sensor 120 is implemented as an electronic sensor, the at least one sensor may be in an OFF state in the OFF state of the sterilization system 100.

According to the disclosure, when the sterilization system 100 is in the OFF state, the controller 130 and/or the sensor 120 is not supplied with power, thereby saving energy provided from a battery of the vehicle 1.

Referring to FIG. 5, based on the storage compartment 10 being switched to a closed state from an open state in the ON state of the sterilization system 100 (Yes in operation 1100), the controller 130 may turn on the lamp module 140 (1200).

Hereinafter, for convenience of description, it is assumed that the operation of turning on the at least one lamp 140-1 and 140-2 is the same as the operation of turning on the lamp module 140.

According to various embodiments, the controller 140 may turn on the lamp module 140 after a preset delay time dt (e.g., approximately 2 seconds) from a point in time that the storage compartment 10 is switched to the closed state from the open state.

As will be described later, the controller 130 may count a duration for which the lamp module 140 is in an ON state, and when a preset condition is satisfied, reset the counted duration.

According to an embodiment, when the lamp module 140 is turned on in a state where the counted duration is reset, the controller 130 may turn on the lamp module 140 after the preset delay time dt from the point in time that the storage compartment 10 is switched to the closed state.

When operating the lamp module 140 immediately when the storage compartment 10 is switched to the closed state from the open state, and if an occupant opens the storage compartment 10 right after closing the storage compartment 10, the occupant is likely to be exposed to UV light.

According to the disclosure, when a sterilization operation is not underway, i.e., when the counted duration is reset, exposure to UV light may be prevented by turning on the lamp module 140 after the preset delay time dt from the point in time that the storage compartment 10 is switched to the closed state.

Also, according to the disclosure, when the occupant forgets to put a personal item in the storage compartment 10 or desires to additionally put the item in the storage compartment 10, the occupant may open the storage compartment 10 to put the item in the storage compartment 10, before the lamp module 140 is operated.

The controller 130 may count the time duration for which the lamp module 140 is in the ON state (1400). For example, the controller 130 may count the duration for which the lamp module 140 is in the ON state, unless the preset condition for turning off the lamp module 140 is satisfied.

For instance, when the storage compartment 10 is not switched to the open state from the closed state (No in operation 1300), the controller 130 may count the duration for which the lamp module 140 is in the ON state.

The controller 130 may count the duration for which the lamp module 140 is in the ON state, and turn off the lamp module 140 (1600) based on the counted duration reaching a preset duration d (Yes in operation 1500).

That is, as long as a preset condition (e.g., opening of the storage compartment 10) for stopping the sterilization process is not satisfied, the controller 130 may operate the lamp module 140 for the preset duration d.

When the counted duration reaches the preset duration d and the lamp module 140 is turned off, the controller 130 may reset the counted duration (1700).

According to various embodiments, when the counted duration is reset, the controller 130 may automatically switch the sterilization system 100 to the OFF state. As another example, when the counted duration is reset, the controller 130 may control a pushlock of the inputter 110 to be released.

According to the disclosure, when the sterilization process is normally completed by the sterilization system 100, an occupant’s intention to sterilize may be reconfirmed by inducing the occupant to operate the inputter 110 again to use the sterilization system 100. Also, according to the disclosure, as the occupant’s intention is reconfirmed, energy wasted by operating the lamp module 140 regardless of the occupant’s intention may be saved.

According to various embodiments, however, even when the counted duration is reset, the controller 130 may maintain the ON state of the sterilization system 100. That is, according to various embodiments, the controller 130 may identify the ON/OFF state of the sterilization system 100 based only on the user input.

The indicator 150 may be lit, based on the lamp module 140 being turned on.

That is, the controller 130 may turn on the indicator 150 based on the lamp module 140 being turned on. Also, the controller 130 may turn off the indicator 150 based on the lamp module 140 being turned off.

That is, the indicator 150 may be lit in a state where the lamp module 140 is turned on, and may not be lit in a state where the lamp module 140 is turned off.

According to the disclosure, when the indicator 150 is lit, the occupant may visually recognize that the sterilization process is being performed by operating the lamp module 140.

The controller 130 may turn off the lamp module 140 (1310), based on the storage compartment 10 being switched to the open state (Yes in operation 1300) in the state where the lamp module 140 is turned on.

According to the disclosure, by turning off the lamp module 140 right after the storage compartment 10 is switched to the open state, UV light may be prevented from being leaked to an outside of the storage compartment 10.

Due to a characteristic where the sterilization system 100 is mounted in the vehicle 1, the storage compartment 10 may be temporarily switched to the open state regardless of an occupant’s operation.

For instance, when a road on which the vehicle 1 is travelling is uneven, the storage compartment 10 may be temporarily detected as the open state due to vibration transmitted to the vehicle 1.

Referring to FIG. 6, based on the storage compartment 10 being switched to the closed state within a preset period of time ot1 (Yes in operation 1320) after the lamp module 140 is turned off (1310) due to the opening of the storage compartment 10 (Yes in operation 1300) in the state where the lamp module 140 is turned on (1200), the controller 130 may turn on the lamp module 140 (1330). When turning on the lamp module 140 (1330), the controller 130 may count a duration for which the lamp module 140 is in the ON state by accumulating the counted duration.

That is, the controller 130 may accumulate a duration d1 for which the lamp module 140 is in the ON state before the OFF state of the lamp module 140, to count the duration for which the lamp module 140 is in the ON state.

In this instance, the preset period of time may be set as a reference time that may be estimated that the storage compartment 10 is temporarily switched to the open state regardless of an occupant’s intention. For example, the preset period of time may be set to approximately 0.5 seconds.

When a sum of the duration d1 for which the lamp module 140 is in the ON state before the OFF state of the lamp module 140 and a duration d2 for which the lamp module 140 is in the ON state after the OFF state of the lamp module 140 reaches the preset duration d (Yes in operation 1500), the controller 130 may turn off the lamp module 140 (1600) and reset the counted duration (1700).

According to various embodiments, when turning on the lamp module 140 (1330), the controller 130 may turn on the lamp module 140 at a point in time that the storage compartment 10 is switched to the closed state.

That is, when turning on the lamp module 140 in a state where the counted duration is not reset, the controller 130 may turn on the lamp module 140 at the point in time that the storage compartment 10 is switched to the closed state.

According to the disclosure, when the storage compartment 10 is temporarily open regardless of the occupant’s intention, the lamp module 140 may be temporarily turned off to minimize UV exposure. However, when the storage compartment 10 is switched back to the closed state, the lamp module 140 may be immediately turned on, thereby completing the sterilization process within a short time.

Also, according to the disclosure, when the storage compartment 10 is temporarily open regardless of the occupant’s intention, the counted duration before the storage compartment 10 is open is summed and determine how far the sterilization process has been performed, thereby completing the sterilization process within a short time.

Referring to FIG. 7, based on a preset period of time ot2 having elapsed (No in operation 1320) without switching to the closed state of the storage compartment 10 after the lamp module 140 is turned off (1310) due to the opening of the storage compartment 10 (Yes in operation 1300) in the state where the lamp module 140 is turned on (1200), the controller 130 may reset the counted duration (1700).

As described above, the preset period of time may be set as a reference time that may be estimated that the storage compartment 10 is temporarily switched to the open state regardless of an occupant’s intention. For example, the preset period of time may be longer than the open time ot1 of FIG. 6 and shorter than the open time ot2 of FIG. 7.

When the preset period of time ot2 is elapsed without switching to the closed state of the storage compartment 10 after the lamp module 140 is turned off (1310) due to the opening of the storage compartment 10 (Yes in operation 1300) in the state where the lamp module 140 is turned on (1200), it may be estimated that the storage compartment 10 is open according to the occupant’s intention. Accordingly, the occupant desires to operate the sterilization process for a new item or temporarily stops the sterilization process.

As described above, according to various embodiments, based on the storage compartment 10 being switched to the closed state from the open state (Yes in operation 1100) in the state where the sterilization system 100 is turned on (Yes in operation 1000), the controller 130 may turn on the lamp module 140 (1200) and count the duration for which the lamp module 140 is in the ON state. Also, the controller 130 may turn off the lamp module 140 based on the counted duration reaching the preset duration d.

Unlike that shown in FIG. 7, according to various embodiments, when the counted duration is reset, the sterilization system 100 may automatically switch the sterilization system 100 to the OFF state, and thus when the preset period of time ot2 is elapsed without switching to the closed state of the storage compartment 10 after the lamp module 140 is turned off (1310) due to the opening of the storage compartment 10 (Yes in operation 1300) in the state where the lamp module 140 is turned on (1200), the controller 130 may automatically switch the sterilization system 100 to the OFF state.

According to the disclosure, when the occupant opens the storage compartment 10 with an intention of adding a new item, an enough sterilization time for the newly stored item may be secured by resetting the counted duration.

In addition, according to the disclosure, when the occupant opens the storage compartment 10 with an intention of removing the item being sterilized, unnecessary power consumption may be prevented by switching the sterilization system 100 to the OFF state.

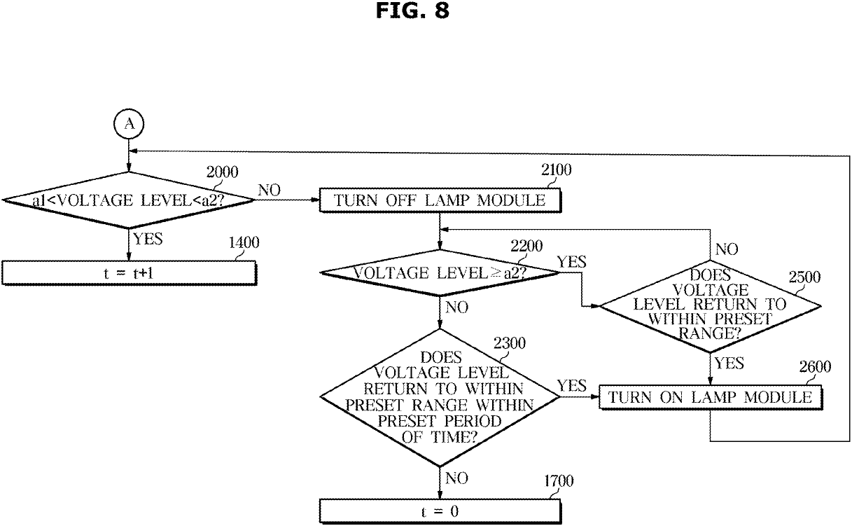

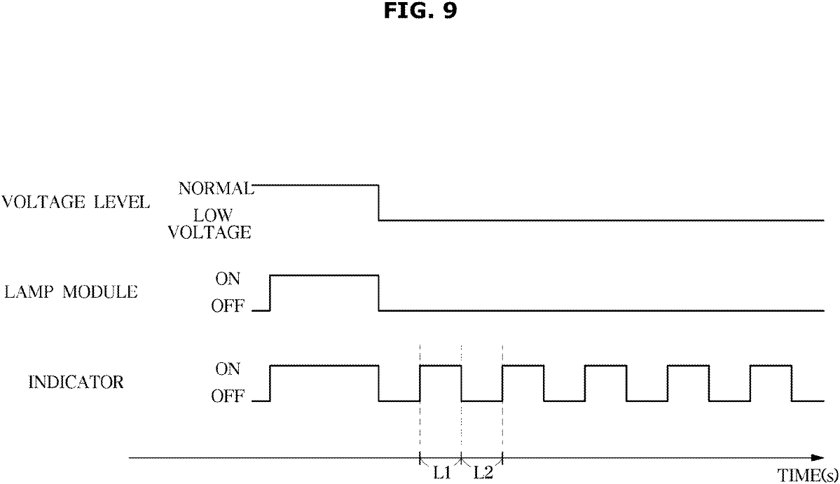

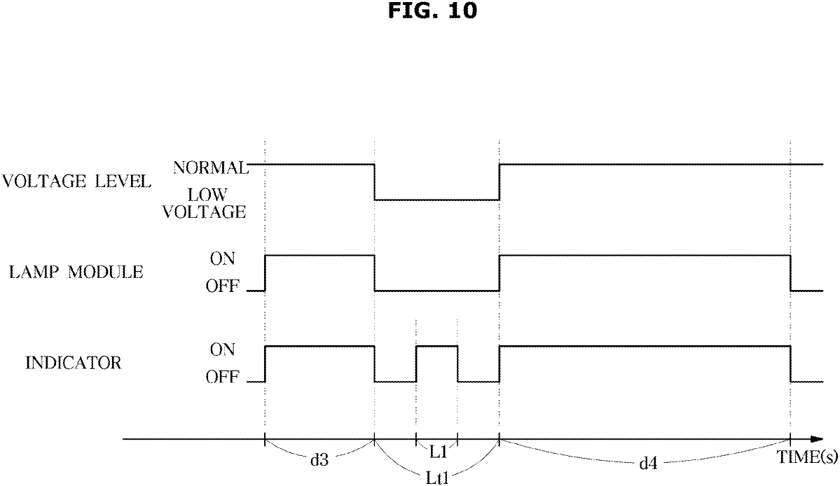

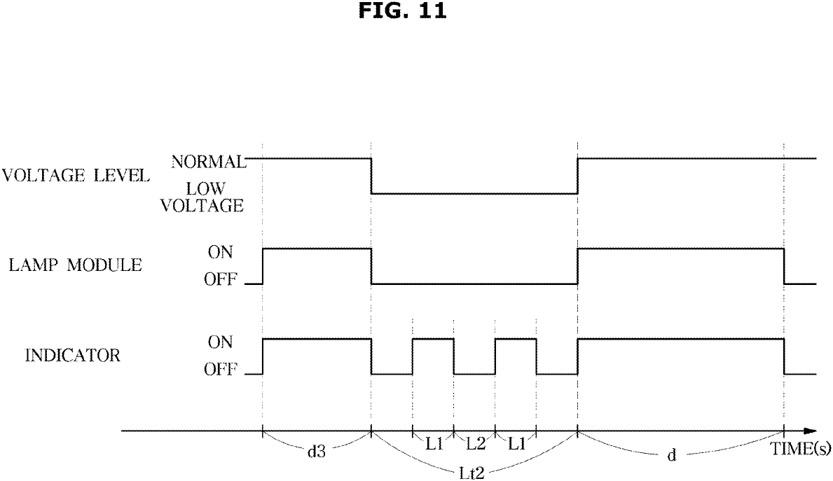

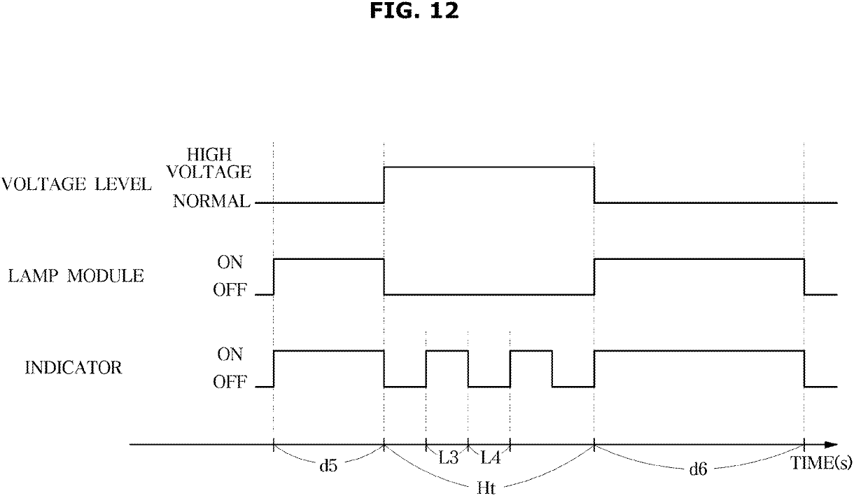

FIG. 8 is a flowchart illustrating another example of a control method of a sterilization system according to an embodiment. FIG. 9 is a diagram illustrating an operation state of each constituent component of a sterilization system when a voltage level supplied to the sterilization system is switched to a low voltage level while sterilization is being performed. FIG. 10 is a diagram illustrating an operation state of each constituent component of a sterilization system when a voltage level supplied to the sterilization system is switched to a low voltage level for a relatively short time while sterilization is being performed. FIG. 11 is a diagram illustrating an operation state of each constituent component of a sterilization system when a voltage level supplied to the sterilization system is switched to a low voltage level for a relatively long time while sterilization is being performed. FIG. 12 is a diagram illustrating an operation state of each constituent component of a sterilization system when a voltage level supplied to the sterilization system is switched to a high voltage level while sterilization is being performed.

Referring to FIG. 8, the controller 130 may determine whether a voltage level of power supplied from a power supply is within a preset range.

The preset range is a reference range for determining whether the voltage level falls within a normal range, and may include a lower limit value a1 and an upper limit value a2.

For example, when the voltage level is lower than the lower limit value a1, the voltage level of the power supplied from the power supply may be determined as a low voltage, and when the voltage level is higher than the upper limit value a2, the voltage level of the power supplied from the power supply may be determined as a high voltage.

When the voltage level of the power supplied from the power supply corresponds to the low voltage, a battery of the vehicle 1 is likely to be discharged. Also, when the voltage level of the power supplied from the power supply corresponds to the high voltage, the lamp module 140 is likely to be damaged.

According to an embodiment, based on the voltage level of the power supplied from the power supply falling within the preset range (Yes in operation 2000), the controller 130 may maintain an ON state of the lamp module 140 and continuously count a duration for which the lamp module 140 is in an ON state (1400).

According to an embodiment, based on the voltage level of the power supplied from the power supply not being within the preset range (No in operation 2000), the controller 130 may turn off the lamp module 140 (2100).

According to the disclosure, when the voltage level of the power supplied from the power supply corresponds to the low voltage, the battery of the vehicle 1 may be prevented from being discharged by turning off the lamp module 140.

Also, according to the disclosure, when the voltage level of the power supplied from the power supply corresponds to the high voltage, a circuit configuration for operating the sterilization system 100 may be prevented from being damaged by turning off the lamp module 140.

When the voltage level corresponds to the low voltage, discharge of the battery of the vehicle 1 is required to be prevented by stopping the operation of the lamp module 140. However, when a sterilization process is finished and a timer is initialized due to a temporary low voltage state, an inconvenience to the occupant may be caused. Accordingly, when the lamp module 140 is turned off due to the temporary low voltage state, there is a need to continue the sterilization process.

Referring to FIG. 9, based on a preset period of time being elapsed after the lamp module 140 is turned off (2100) due to a decrease in the voltage level below a preset level a1 (No in operation 2000, No in operation 2200) in a state where the lamp module 140 is turned on, the controller 130 may reset a counted duration (1700).

In this instance, the preset period of time may be set as a reference time for determining whether a low voltage state is temporary. For example, the preset period of time may be set to approximately 2 seconds.

According to various embodiments, a first time, which is a reference time that it is estimated that the storage compartment 10 is temporarily switched to an open state regardless of an occupant’s intention, is different from a second time which is a reference time for determining whether a low voltage state is temporary. For example, the first time may be shorter than the second time.

Also, based on the voltage level being equal to or less than the preset level a1, the controller 130 may control the indicator 150 to provide a first visual feedback.

The first visual feedback may include a first blinking pattern. For example, the first blinking pattern may include a blinking pattern having an ON/OFF cycle of L1/L2.

According to the disclosure, when the low voltage state of the power supplied from the power supply is not temporary, death of the battery of the vehicle 1 may be efficiently prevented by ending the sterilization process.

Referring to FIG. 10, based on the voltage level returning to within the preset range within a preset period of time Lt1 (Yes in operation 2300) after the lamp module 140 is turned off (2100) due to the decrease in the voltage level below the preset level a1 (No in operation 2000, No in operation 2200) in the state where the lamp module 140 is turned on, the controller 130 may turn on the lamp module 140 and count a duration for which the lamp module 140 is in the ON state by accumulating the counted duration (2600). According to various embodiments, the controller 130 may control the indicator 150 to blink according to the first blinking pattern for a duration when the voltage level is equal to or less than the preset level a1.

As described above, when a sum of a duration d3 for which the lamp module 140 is in the ON state before the OFF state of the lamp module 140 and a duration d4 for which the lamp module 140 is in the ON state after the OFF state of the lamp module 140 reaches the preset duration d (Yes in operation 1500), the controller 130 may turn off the lamp module 140 (1600) and reset the counted duration (1700).

According to the disclosure, when the low voltage state of the power supplied from the power supply is temporary, occupant’s inconvenience may be prevented by restarting the sterilization process without stopping the sterilization process.

Referring to FIG. 11, according to various embodiments, based on the voltage level returning to within the preset range after the counted duration is reset according to an elapse of a preset period of time Lt2 after the lamp module 140 is turned off (2100) due to the decrease in the voltage level below the preset level a1 (No in operation 2000, No in operation 2200) in the state where the lamp module 140 is turned on, the controller 130 may turn on the lamp module 140 (1200) and count a duration for which the lamp module 140 is in the ON state. Also, the controller 130 may turn off the lamp module 140 based on the counted duration reaching the preset duration d.

In addition, the controller 130 may control the indicator 150 to blink in the first blinking pattern for the period of time Lt2 that the voltage level is equal to or less than the preset level a1.

By contrast, unlike that shown in FIG. 11, according to various embodiments, when the counted duration is reset, the sterilization system 100 may automatically switch the sterilization system 100 to an OFF state, and thus when the preset period of time Lt2 is elapsed after the lamp module 140 is turned off (2100) due to the decrease in the voltage level below the preset level a1 (No in operation 2000, No in operation 2200) in the state where the lamp module 140 is turned on, the controller 130 may automatically switch the sterilization system 100 to the OFF state.

According to the disclosure, when the low voltage state of the power supplied from the power supply is relieved, the occupant’s inconvenience may be prevented by restarting the sterilization process without the occupant’s operation.

Meanwhile, in general, because a high voltage state is temporary and is not related to discharge of the battery of the vehicle 1. Accordingly, when the voltage level returns to within the normal range, it is preferable to restart the sterilization process.

Referring to FIG. 12, when the lamp module 140 is turned off (2100) due to an increase in the voltage level above a preset level a2 (No in operation 2000, Yes in operation 2200) in the state where the lamp module 140 is turned on, the controller 130 may maintain the counted duration until the voltage level returns to within the preset range (No in operation 2500).

In other words, based on the voltage level being equal to or greater than the preset level a2, the controller 130 may stand by until the voltage level returns to within the preset range without resetting the counted duration.

That is, the controller 130 may not reset the counted duration regardless of a length of duration Ht that the voltage level increases above the preset level a2.

Based on the voltage level returning to within the preset range (Yes in operation 2500), the controller 130 may turn on the lamp module 140 and count the duration for which the lamp module 140 is in the ON state by accumulating the counted duration (2600).

As described above, when a sum of a duration d5 for which the lamp module 140 is in the ON state before the OFF state of the lamp module 140 and a duration d6 for which the lamp module 140 is in the ON state after the OFF state of the lamp module 140 reaches the preset duration d (Yes in operation 1500), the controller 130 may turn off the lamp module 140 (1600) and reset the counted duration (1700).

According to the disclosure, when the voltage level of the power supplied from the power supply corresponds to a high voltage, the sterilization process may be performed after standing by until the high voltage state is relieved, thereby preventing the lamp module 140 from being damaged and efficiently completing the sterilization process.

Also, based on the voltage level being equal to or greater than the preset level a2, the controller 130 may control the indicator 150 to provide a second visual feedback.

The second visual feedback may include a second blinking pattern. For example, the second blinking pattern may include a blinking pattern having an ON/OFF cycle of L3/L4.

The first visual feedback, provided by the indicator 150 when the voltage level is equal to or less than the preset level a1, may be different from the second visual feedback provided by the indicator 150 when the voltage level equal to or greater than the preset level a2. For example, the ON/OFF cycle of L1/L2 of the first blinking pattern may be different from the ON/OFF cycle of L3/L4 of the second blinking pattern.

According to the disclosure, the occupant may easily identify whether a cause that the lamp module 140 is not operated is the low voltage state or high voltage state, and take action depending on a corresponding state.

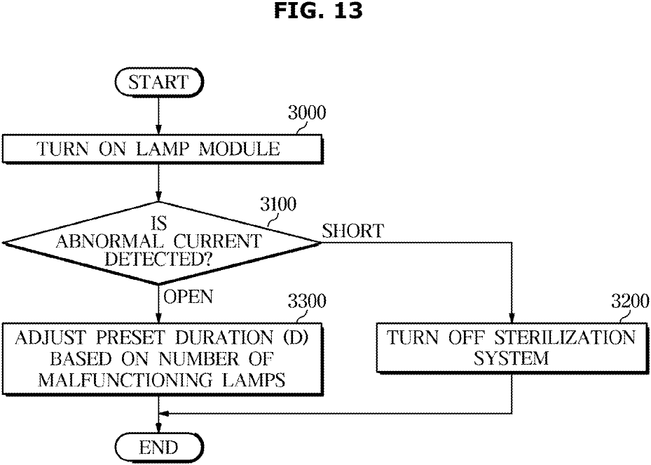

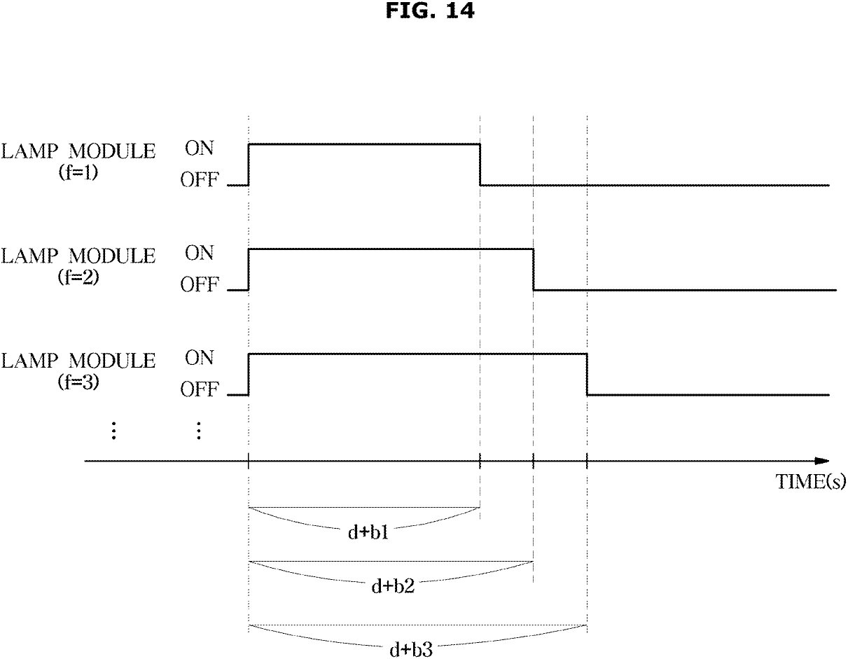

FIG. 13 is a flowchart illustrating still another example of a control method of a sterilization system according to an embodiment. FIG. 14 illustrates an example where a sterilization operation duration is changed depending on a number of malfunctioning lamps.

Referring to FIG. 13, the controller 130 may apply current to the lamp module 140 (3000).

For example, based on the storage compartment 10 being switched to a closed state from an open state in a state where the sterilization system 100 is turned on, the controller 130 may apply the current to the lamp module 140.

As another example, even when a counted duration is reset and a sterilization process ends, the controller 130 may periodically apply the current to the lamp module 140 based on the storage compartment 10 being in the closed state.

As still another example, even when the sterilization system 100 is in an OFF state, the controller 130 may periodically apply the current to the lamp module 140 based on the storage compartment 10 being in the closed state.

The controller 130 may detect a failure state of the plurality of lamps 140-1 and 140-2 included in the lamp module 140, based on the current applied to the plurality of lamps 140-1 and 140-2.

For instance, based on driving current for driving the plurality of lamps 140-1 and 140-2 being significantly large (e.g., a short-circuited state) or significantly small (e.g., an open state), the controller 130 may detect the failure state of the plurality of lamps 140-1 and 140-2.

Based on abnormal current being detected by the second sensor that detects the current applied to the plurality of lamps, the controller 130 may identify the failure state of the plurality of lamps 140-1 and 140-2 according to a magnitude of the abnormal current.

For example, when a current value detected from the second sensor is greater than a first preset value, the controller 130 may determine that an electrical wire connected to at least one lamp is short-circuited.

When the electrical wire connected to one lamp 140-1 of the plurality of lamps is short-circuited, a state of the lamp 140-1 whose electrical wire is short-circuited may be defined as the short-circuited state.

When at least one of the plurality of lamps is in the short-circuited state, all components of the sterilization system 100 are likely to be damaged due to an overcurrent. Accordingly, it is preferable not to apply current to the lamp module 140.

Based on the at least one of the plurality of lamps being determined as the short-circuited state (SHORT in operation 3100), the controller 130 may switch the sterilization system 100 to an OFF state regardless of an user input through the inputter 110 (3200).

Also, based on the at least one of the plurality of lamps being determined as the short-circuited state, the controller 130 may control the indicator 150 to provide a third visual feedback.

The third visual feedback may include a third blinking pattern. For example, the third blinking pattern may be different from the first and second blinking patterns.

According to the disclosure, when an overcurrent is detected, the OFF state of the sterilization system 100 may be maintained regardless of the user input, thereby preventing a circuit component from being damaged due to the overcurrent.

In addition, according to the disclosure, when the overcurrent is detected, the third visual feedback, different from the first and second visual feedbacks, may be provided, thereby allowing the user to take action quickly.

As another example, when a current value detected by the second sensor is lower than a second preset value, the controller 130 may determine that an electrical wire connected to at least one lamp is opened or shorted. Further, the controller 130 may determine the number of lamps which are in an open state according to the current value detected by the second sensor.

When current is not supplied to the electrical wire connected to one lamp 140-1 of the plurality of lamps, a state of the one lamp 140-1 may be defined as an open state.

Because a lamp in the open state is not operated, when the at least one lamp corresponds to the open state, an efficiency of sterilization may be reduced.

When the at least one lamp 140-1 of the plurality of lamps 140-1 and 140-2 is determined as the open state and the other lamp 140-2 is determined as a normal state (OPEN in operation 3100), the controller 130 may adjust a preset duration d based on the number of lamps (f) determined as the open state (3300).

For example, referring to FIG. 14, when the number of lamps (f) determined as the open state among the plurality of lamps included in the lamp module 140 is one, the preset duration may be adjusted to a value obtained by adding a first preset duration b1 to the default duration d.

Also, when the number of lamps (f) determined as the open state among the plurality of lamps included in the lamp module 140 is two, the preset duration may be adjusted to a value obtained by adding a second preset duration b2 to the default duration d.

In addition, when the number of lamps (f) determined as the open state among the plurality of lamps included in the lamp module 140 is three, the preset duration may be adjusted to a value obtained by adding a third preset duration b3 to the default duration d.

In this instance, the third preset duration b3 may be longer than the second preset duration b2 and the second preset duration b2 may be longer than the first preset duration b1.

That is, the controller 130 may increase the preset duration as the number of lamps determined as the open state increases.

Also, based on the at least one lamp among the plurality of lamps being determined as the open state, the controller 130 may control the indicator 150 to provide a fourth visual feedback.

The fourth visual feedback may include a fourth blinking pattern. For example, the fourth blinking pattern may be different from the first, the second and the third blinking patterns.

According to the disclosure, when the least one lamp of the plurality of lamps included in the lamp module 140 is not operated, a constant sterilization effect may be maintained by increasing a sterilization period.

According to various embodiments, the controller 130 may perform a test run for determining whether the sterilization system 100 is in an operable state at various point in times, such as when the vehicle 1 is started and/or when the storage compartment 10 is switched to a closed state after the vehicle 1 is started and the sterilization system 100 is turned on for the first time.

During the test run, the controller 130 may determine whether a voltage level of power supplied from the power supply falls within a preset range, and determine whether the lamp module 140 is in the short-circuited state or open state.

According to various embodiments, the controller 130 may control the indicator 150 to blink in a blinking pattern corresponding to a state of the sterilization system 100.

For example, based on the voltage level being equal to or less than the preset level a1, the controller 130 may control the indicator 150 to provide the first visual feedback. Based on the voltage level being equal to or greater than the preset level a2, the controller 130 may control the indicator 150 to provide the second visual feedback. Based on at least one of a plurality of lamps being determined as the short-circuited state, the controller 130 may control the indicator 150 to provide the third visual feedback. Based on at least one of a plurality of lamps being determined as the open state, the controller 130 may control the indicator 150 to provide the fourth visual feedback.

In this instance, the first to fourth visual feedbacks may be different from each other.

According to the disclosure, an occupant may intuitively identify a state of the sterilization system 100 according to the visual feedback provided by the indicator 150, and take action depending on the state of the sterilization system 100.

Meanwhile, a portion of constituent components of the sterilization system 100 may comprise a software component and/or a hardware component, such as a field programmable gate array (FPGA) and an application specific integrated circuit (ASIC).

As is apparent from the above, according to the disclosure, the sterilization system and the control method thereof can allow an occupant to easily sterilize a personal item.

According to the disclosure, the sterilization system and the control method thereof can prevent a risk of vehicle occupant’s exposure to UV.

According to the disclosure, the sterilization system and the control method thereof can sterilize an occupant’s personal item with optimum energy efficiency.

According to the disclosure, the sterilization system and the control method thereof can prevent a sterilization operation from being stopped due to a movement of a vehicle regardless of an occupant’s intention.

According to the disclosure, the sterilization system and the control method thereof can allow an occupant to easily check a state of the sterilization system.

Meanwhile, the disclosed embodiments may be embodied in the form of a recording medium storing instructions executable by a computer. The instructions may be stored in the form of program code and, when executed by a processor, may generate a program module to perform the operations of the disclosed embodiments. The recording medium may be embodied as a computer-readable recording medium.

The computer-readable recording medium includes all kinds of recording media in which instructions which may be decoded by a computer are stored, for example, a read only memory (ROM), a random access memory (RAM), a magnetic tape, a magnetic disk, a flash memory, an optical data storage device, and the like.

Although embodiments of the disclosure have been described for illustrative purposes, those skilled in the art will appreciate that various modifications, additions and substitutions are possible, without departing from the scope and spirit of the disclosure. Therefore, embodiments of the disclosure have not been described for limiting purposes.

1. A sterilization system that sterilizes an interior of a storage compartment of a vehicle, comprising:
at least one lamp configured to irradiate ultraviolet light to the interior of the storage compartment;
a sensor configured to detect an open or closed state of the storage compartment; and
a controller configured to:
based on the storage compartment being switched from an open state to a closed state in a state where the sterilization system is turned on, turn on the at least one lamp,
count a duration for which the at least one lamp is in an ON state,
based on the counted duration reaching a preset duration, turn off the at least one lamp, and
based on the storage compartment being switched to the open state in a state where the at least one lamp is turned on, turn off the at least one lamp.

2. The sterilization system of claim 1, wherein, based on the storage compartment being switched to the closed state within a preset period of time after the at least one lamp is turned off due to an opening of the storage compartment, the controller is configured to turn on the at least one lamp and accumulate the counted duration to count the duration for which the at least one lamp is in the ON state.

3. The sterilization system of claim 2, wherein the controller is configured to reset the counted duration, based on the preset period of time being elapsed after the at least one lamp is turned off due to the opening of the storage compartment.

4. The sterilization system of claim 3, wherein the controller is configured to:
when turning on the at least one lamp in a state where the counted duration is not reset, turn on the at least one lamp at a point in time that the storage compartment is switched to the closed state, and
when turning on the at least one lamp in a state where the counted duration is reset, turn on the at least one lamp after a delay time from the point in time that the storage compartment is switched to the closed state.

5. The sterilization system of claim 1, further comprising:
a power supply configured to supply power supplied from a battery of the vehicle to the sterilization system,
wherein the controller is configured to turn off the at least one lamp based on a voltage level of the power supplied from the power supply being out of a preset range.

6. The sterilization system of claim 5, wherein, based on the voltage level returning to within the preset range within a preset period of time after the at least one lamp is turned off due to a decrease in the voltage level below a preset level, the controller is configured to turn on the at least one lamp and accumulate the counted duration to count the duration for which the at least one lamp is in the ON state.

7. The sterilization system of claim 6, wherein the controller is configured to reset the counted duration, based on the preset period of time being elapsed after the at least one lamp is turned off due to the decrease in the voltage level below the preset level.

8. The sterilization system of claim 5, wherein the controller is configured to:
when the at least one lamp is turned off due to an increase in the voltage level above a preset level, maintain the counted duration until the voltage level returns to within the preset range, and
based on the voltage level returning to within the preset range, turn on the at least one lamp and accumulate the counted duration to count the duration for which the at least one lamp is in the ON state.

9. The sterilization system of claim 5, further comprising:
an indicator configured to provide a visual feedback related to a state of the sterilization system,
wherein the controller is configured to control the indicator to provide a first visual feedback based on the voltage level being equal to or less than a first preset level, and control the indicator to provide a second visual feedback based on the voltage level being equal to or greater than a second preset level,
the first preset level is a lower limit value of the preset range, the second preset level is an upper limit value of the preset range, and the first visual feedback and the second visual feedback are different from each other.

10. The sterilization system of claim 1, wherein the at least one lamp comprises a plurality of lamps, and the controller is configured to detect a failure state of the plurality of lamps based on a current applied to the plurality of lamps.

11. The sterilization system of claim 10, wherein the controller is configured to switch the sterilization system to an OFF state, based on at least one lamp of the plurality of lamps being determined as a short-circuited state.

12. The sterilization system of claim 10, wherein, when at least one lamp of the plurality of lamps is determined as an open state and other lamps of the plurality of lamps are determined as a normal state, the controller is configured to adjust the preset duration based on a number of lamps determined as the open state.

13. The sterilization system of claim 10, further comprising:
an indicator configured to provide a visual feedback related to a state of the sterilization system,
wherein the controller is configured to control the indicator to provide a first visual feedback based on at least one lamp of the plurality of lamps being determined as a short-circuited state, and control the indicator to provide a second visual feedback based on at least one lamp of the plurality of lamps being determined as an open state, and
the first visual feedback and the second visual feedback are different from each other.

14. The sterilization system of claim 1, further comprising:
an indicator configured to provide a visual feedback related to a state of the sterilization system,
wherein the controller is configured to turn on the indicator based on the at least one lamp being turned on.

15. The sterilization system of claim 1, further comprising:
an inputter configured to receive a user input for switching the sterilization system to an ON state or an OFF state,
wherein the inputter is provided inside the storage compartment.

16. A control method of a sterilization system that sterilizes an interior of a storage compartment of a vehicle, the control method comprising:
based on the storage compartment being switched from an open state to a closed state in a state where the sterilization system is turned on, turning on at least one lamp that irradiates ultraviolet light to the interior of the storage compartment,
counting a duration for which the at least one lamp is in an ON state,
based on the counted duration reaching a preset duration, turning off the at least one lamp, and
based on the storage compartment being switched to the open state in a state where the at least one lamp is turned on, turning off the at least one lamp.

17. The control method of claim 16, further comprising:
based on the storage compartment being switched to the closed state within a preset period of time after the at least one lamp is turned off due to an opening of the storage compartment, turning on the at least one lamp and accumulating the counted duration to count the duration for which the at least one lamp is in the ON state.

18. The control method of claim 17, further comprising:
resetting the counted duration, based on the preset period of time being elapsed after the at least one lamp is turned off due to the opening of the storage compartment.

19. The control method of claim 18, further comprising:
when turning on the at least one lamp in a state where the counted duration is not reset, turning on the at least one lamp at a point in time that the storage compartment is switched to the closed state, and
when turning on the at least one lamp in a state where the counted duration is reset, turning on the at least one lamp after a delay time from the point in time that the storage compartment is switched to the closed state.

20. The control method of claim 16, further comprising:
turning off the at least one lamp, based on a voltage level of power supplied from a power supply being out of a preset range, the power supply configured to supply power supplied from a battery of the vehicle to the sterilization system.
